(19) United States
(12) Patent Application Publication
(10) Pub. No.: US 2025/0161520 A1
(43) Pub. Date: May 22, 2025

(54) DEVICE FOR AIR TREATMENT BY MEANS OF UV RADIATION AND ASSEMBLY WITH THIS DEVICE

(71) Applicant: OSRAM GmbH, Muenchen (DE)

(72) Inventor: Ulrich Hartwig, Berlin (DE)

(21) Appl. No.: 18/834,782

(22) PCT Filed: Dec. 7, 2022
(86) PCT No.: PCT/EP2022/084707

§ 371 Date: Jul. 31, 2024

(30) Foreign Application Priority Data
Feb. 4, 2022 (DE) 10 2022 201 206.2

(51) Int. Cl. A61L 9/20

(52) U.S. Cl. CPC A61L 9/20; A61L 2209/12

(57) ABSTRACT

In an embodiment a device includes at least one UV radiation source configured to emit radiation in a range of ultraviolet wavelengths and a beam-shaping and collimating optics configured to shape the radiation to a UV beam at least approximately collimated in at least one dimension, wherein a beam angle, defined as an angle to an optical axis of the UV beam including 99% of a radiant flux is smaller than 2°.

### CROSS-REFERENCE TO RELATED APPLICATIONS

This patent application is a national phase filing under section 371 of PCT/EP2022/084707, filed Dec. 7, 2022, which claims the priority of German patent application 10 2022 201 206.2, filed Feb. 4, 2022, each of which is incorporated herein by reference in its entirety.

### TECHNICAL FIELD

The present disclosure relates to a device for air treatment by means of ultraviolet (UV) radiation, particularly for disinfection, and an assembly for air treatment in a room comprising such a device.

### BACKGROUND

Such type of devices for air treatment are designed for the treatment of the air in rooms to improve the indoor air quality. Preferably, the UV radiation used extends into the UV-C region, i.e. wavelengths in the range from 280 nm down to 100 nm, because of the germicidal effectivity of UV radiation in this wavelength range. However, UV-C radiation is hazardous for eyes and skin. For safety reasons, it must be assured that the UV radiation does not harm any person entering the room or staying therein. Therefore, such devices are typically mounted either at the ceiling of the room or at least as close beneath the ceiling as possible, e.g. on a wall at a position close beneath the ceiling. For this reason, such devices are also referred to as Upper Air Germicidal Ultraviolet (GUV) devices.

In an upper active zone, the minimum UV radiance should typically be more than 10 μW/cm² to ensure effective air treatment. Below the active zone the safe zone is located. In this safe zone the UV irradiance should be below 1 mW/m². This low level of UV irradiance allows people to stay longer in the safe zone, e.g. 8 hours. The transition zone forms the transition between active zone and safe zone with a high gradient of irradiations.

If the active zone is only confined partially by the ceiling and the walls, functioning as the only beam-dump, the maximum radiation power is limited to keep the irradiance in the safe zone below 1 mW/m². For example, assuming homogeneous irradiation of a ceiling area of 100 m² and Lambertian reflecting properties with a reflectivity of 0.1, the maximum radiation power is only 1 W.

Therefore, additional measures are usually necessary to assure that any UV radiation leaking from the GUV device does not exceed a safety threshold inside a safety zone below the GUV device but is confined inside the active zone as good as possible.

In current GUV devices UV-C low pressure lamps are used. Because of their relatively spacious size and tubular shape the etendue of such UV-C lamps is very large and, consequently, the UV-C radiance is relatively small. To confine the UV-C radiation inside the active zone and ensure a safe zone in the space below the ceiling, reflectors and baffles or louvers are used. The latter is designed to absorb all the UV-C radiation unintentionally reflected towards the safe zone, resulting in efficiencies which are usually below 10%.

### SUMMARY

Embodiments provide an improved device for air treatment.

Further embodiments provide a device for air treatment with improved efficiency and lower power consumption.

Yet further embodiments provide a device for air treatment with improved UV radiation safety.

Yet other embodiments provide an assembly for improved air treatment, comprising said device for air treatment.

Embodiments and developments of the device for air treatment and the assembly comprising the device for air treatment are defined as the respective dependent claims and are the subject matter of the description and the drawings.

Various implementations and embodiments of the device for air treatment disclosed herein comprise a UV radiation source and a beam-shaping and collimating optics configured to shape the radiation emitted by the UV radiation source to a beam at least approximately collimated in at least one dimension.

In the configuration comprising a beam collimated only in one dimension (1D-collimation configuration), the collimation of the beam extends only in one direction of space, hereinafter referred to as 1D-collimation axis. The 1D-collimation axis is perpendicular to the main direction of the beam. As a result, there is at least one projection plane in which the perpendicularly projected rays of the beam are approximately parallel to a perpendicularly projected main direction of the one-dimensionally collimated beam. In other words, the UV rays of the 1D-collimated beam are approximately parallel only when viewed in the viewing direction that is perpendicular to the 1D-collimation axis and the main direction of the beam.

In a 1D-collimation configuration, the UV radiation source and the beam-shaping and collimating optics may further be configured to shape the beam to have a wide-angle distribution with respect to the second direction of space. In other words, the UV rays of the 1D-collimated beam are divergent when viewed in the viewing direction that is perpendicular to the second direction of space and the main direction of the beam.

In the context of the present disclosure, the term “approximately parallel” may mean that the beam divergence in the projection plane is small, typically only a few degrees (half angle), for example, smaller than 2°, more preferably smaller than 1° or even smaller than 0.5°.

Furthermore, the UV radiation source and the beam-shaping and collimating optics may further be configured to at least approximately collimate the UV radiation in two dimensions (2D-collimation configuration). In the 2D-collimation configuration, the collimation of the beam extends also in a second direction of space, hereinafter referred to as the 2D-collimation axis. The 2D-collimation axis is perpendicular to the 1D-collimation axis and to the main direction of the beam. As a result, in a second projection plane that is perpendicular to the first projection plane and parallel to the main direction of the two-dimensionally collimated beam the perpendicularly projected rays of the beam are also approximately parallel. In other words, in this configuration the rays are not only parallel when projected in a specific projection plane, but essentially all the rays of the beam are at least approximately parallel thereby defining the direction of the two-dimensionally (2D-) collimated beam.

Particularly, the UV radiation source has a small etendue compared to conventional UV radiation sources, e.g. tubular, T5 diameter, low pressure mercury vapor discharge lamps that produce broadband UV-C at 253.7 nm.

Particularly suitable UV radiation sources comprise UV(-C) light emitting diodes (LEDs) with high radiance or UV(-C) laser diodes (LDs). LEDs have a small etendue, e.g. approximately 3.14 mm²sr for a 1 mm² chip size without immersed optics.

The etendue of the beam of known upper-air GUV devices is usually between 500 mm²sr to 20,000 mm²sr, depending on the respective device's geometry and angle of radiation. In comparison, using a radiation source with a small etendue, e.g. an LED, offers plenty of leeway for the optical design of the device. Furthermore, larger-size LEDs and even arrays of multiple LEDs or LDs may be suitable, if arranged in a compact form factor, i.e. a point-like source or a line-like source. The former is preferred for 2D-collimated beam configurations. The latter may be suitable for 1D-collimated beam configurations, i.e. in which the far field beam angle needs to be small or approximately collimated only in one direction. Therefore, the geometrical dimensions of the emitting area of the UV radiation source also needs to be small only in one direction, e.g. smaller than 2 mm, preferably smaller than 1 mm. In the second direction the source may extend several centimeters or even meters. For example, a linear array of single LEDs is suitable for 1D-collimated beam configurations.

Moreover, when using small etendue sources the UV radiation can be shaped more accurately defined by means of optical elements and then directed precisely several times through the available space in the active zone by means of reflectors, without the risk of spreading the UV radiation towards the safe zone. At the end of multiple reflection passes, i.e. at the end of the reflection-chain, the radiation is either consumed by losses of the reflector installation or by a defined beam-dump (or both).

The device for air treatment disclosed herein further comprises a beam-shaping and collimating optics that is configured to collimate the radiation emitted by the UV radiation source in at least one dimension. Particularly, the beam-shaping and collimating optics is configured such that the beam angle φ_{99%} is small, preferably smaller than 2°, more preferably smaller than 1° or even smaller than 0.5° to keep the radiation within the active zone, whereas the angle φ_{99%} is defined by the radiant flux content according to the following equations:

$$\begin{matrix}{Φ}_{99 \%} = {\int }_{- {φ}_{99 \%}}^{{φ}_{99 \%}} {\int }_{0}^{180 °} I ( γ , φ ) \cdot sin γ d γ d φ \\ Φ = {\int }_{- 180 °}^{180 °} {\int }_{0}^{180 °} {\int }_{0}^{180 °} I ( γ , φ ) \cdot sin γ d θ d φ : total radiant flux\end{matrix}$$

I(γ, φ): function radiant intensity depending on the angles γ and φ
   ¢99%: 99% of the total radiant flux ¢
γ: angle to the optical axis (z-axis) in the y-z-plane
φ: angle to the optical axis (z-axis) in the x-z-plane
φ_{99%}: angle to the optical axis (z-axis) in the x-z-plane
   including 99% of the total radiant flux

Suitable beam-shaping and collimating optics may comprise, inter alia, reflectors known for use with small etendue, i.e. point-like radiation sources, such as parabolic or elliptical reflectors, freeform reflectors, lenses, TIR-lenses and combinations thereof. The shape of the reflector may also differ in two perpendicular planes to particularly allow for a collimated UV beam in the vertical direction and a wide-angle beam in the horizontal direction within the active zone. In various preferred implementations and embodiments, the small etendue radiation source comprises at least one UV-LED or UV-LD. Furthermore, the at least one UV-LED or UV-LD may comprise a primary optic configured to function as an element of the beam-shaping and collimating optics. For more details see the description of specific embodiments below.

The combination of a radiation source having a small etendue and a beam-shaping and collimating optics enable effective beam-shaping and collimating of the UV radiation, at least in one dimension. As an advantageous result, a defined narrow active zone with a typical vertical expansion of approximately 20 cm to 80 cm can be achieved, in which the UV irradiance is high enough for effective reduction of microorganisms like fungal cells, fungal spores, bacteria, bacteria spores and viruses.

In various implementations and embodiments of the assembly for air treatment, an active zone, in which the UV radiation beam is confined, is implemented in the upper part of a room.

A basic embodiment of the assembly for air treatment (single pass configuration) essentially consists of a device for air treatment described above and a beam-dump. The device for air treatment is arranged in an upper part of a room in such a manner that the rays of the UV radiation beam propagate approximately collimated when viewed in the horizontal viewing direction. When viewed in the vertical viewing direction the rays may propagate approximately uncollimated (1D-collimated configuration) or also approximately collimated (2D-collimated configuration). In any case, the UV radiation is optically confined in the active zone in the upper part of the room, because the UV radiation beam, emerging from the GUV device, is vertically collimated. A beam-dump, arranged vis-à-vis the GUV device in a distance along the main direction of the UV radiation beam, collects and absorbs the UVC-radiation at the dead end of the active zone. In other words, the distance between the GUV device and the beam-dump defines the downstream end of the UV radiation beam and, thus, the longitudinal extension of the active zone. Preferably, the GUV device is configured such that the main direction of the UV radiation beam is oriented at least approximately in a horizontal direction. Thereby, the active zone is horizontally oriented, which ensures the same safe distance between the lower periphery of the active zone and people moving in the safe zone of the room beneath the active zone.

In a preferred embodiment of the assembly for air treatment (multiple pass configuration), the assembly comprises at least one device for air treatment described above and further comprises one or more external reflectors. The external reflectors are arranged and configured to reflect the UV radiation multiple times through the available space of the active zone. For this purpose, the external reflectors are appropriately arranged outside the device for air treatment and at longitudinal boundaries of the active zone. For example, a first external reflector may be arranged vis-à-vis the GUV device in a distance along the main direction of the UV radiation beam coming from the GUV device, thereby defining the longitudinal extension of the active zone in between. Furthermore, the first external reflector is configured to reflect the UV radiation beam coming from the GUV device (first pass) back through the active zone and towards the GUV device (second pass).

In some enhanced embodiments of the assembly, a second external reflector or appropriate mirror area and even a third, fourth, etc. reflect the UV radiation beam back and forth (third pass, fourth pass, etc.), e.g. in a zigzag shape extending transversal within the active zone, essentially in a horizontal direction. In order to enable such multiple passes of the UV radiation beam through the active zone separate external reflector are arranged and configured appropriately, i.e. with proper alignment and/or shape of the mirror surface. Alternatively, the mirror area of a reflector may be elongated in the horizontal direction to match the horizontal extension of the active zone. Advantageously, multiple passes of the UV radiation increase the irradiation inside the active zone and, hence, the germicidal air treatment. Preferably, at the downstream end of the chain of multiple reflections the UV radiation beam is finally safely collect and absorbed by beam-dumps with a defined low reflectance.

In some embodiments, the assembly for air treatment further comprises beam-dumps installed in a transition zone directly above and/or below the active zone, e.g. above and/or below the external reflector(s) and/or the device for air treatment. The beam-dumps are arranged and configured to absorb the residual radiation leaking from the active zone, e.g. stray radiation.

By this means, the device for air treatment disclosed herein can safely be installed even in rooms with a low ceiling. Furthermore, the defined optical confinement of the UV radiation within the active zone also enables installing additional devices above the active zone like luminaires, air cons, smoke detectors, etc.

According to at least one embodiment of the assembly for air treatment, the border of the active zone can be equipped with fluorescent material to visually indicate UV radiation leaking from the active zone and/or indicating operation of the device.

Furthermore, electrical sensors, e.g. UV photodiodes, may be installed to indicate and measure radiation spilling out of the active zone. The signal of such sensors can be used to shut off the device for air treatment if too much radiation leaks from the active zone. Also, the signal can be used to indicate that a service for the device or the external reflectors, e.g. cleaning, are needed.

Some embodiments further comprise a fan configured to circulate the treated air from the active zone into the safe zone, the air from the safe zone into the active zone, etc. by means of forced convection. By this means, effective treatment of the air volume of the whole room is improved. The fan may also be used for cooling the UV LEDs.

### BRIEF DESCRIPTION OF THE DRAWINGS

These and other aspects of the invention will now be further described, by way of exemplary embodiments only, with reference to the accompanying figures, in which:

FIG. 1A shows a side view of a first embodiment of a device for air treatment;

FIG. 1B shows a top view of the embodiment shown in FIG. 1A;

FIG. 2A shows a side view of a second embodiment of a device for air treatment;

FIG. 2B shows a top view of the embodiment shown in FIG. 2A;

FIG. 3 shows a side view of a first embodiment of an assembly for air treatment;

FIG. 4 shows a side view of a second embodiment of an assembly for air treatment;

FIG. 5 shows a top view of a third embodiment of an assembly for air treatment;

FIG. 6 shows a partial top view of a fourth embodiment of an assembly for air treatment;

FIG. 7 shows a partial top view of a fifth embodiment of an assembly for air treatment;

FIG. 8 shows a partial top view of a sixth embodiment of an assembly for air treatment;

FIG. 9 shows a side view of an example of an external reflector, having a flat reflective surface;

FIG. 10 shows a side view of another example of an external reflector, having a parabolic reflective surface;

FIG. 11 shows a side view of third example of an external reflector, having an elliptical or spherical reflective surface;

FIG. 12 shows a side view of an external reflector with louvers;

FIG. 13 shows a side or top view of an example of a beam dump; and

FIG. 14 shows a side or top view of another example of a beam dump.

Like elements are indicated by like reference numerals. Identical or essentially identical elements may be described only with respect to the figures where they are shown first. They may not be reiterated in the description of successive figures.

### DETAILED DESCRIPTION OF ILLUSTRATIVE EMBODIMENTS

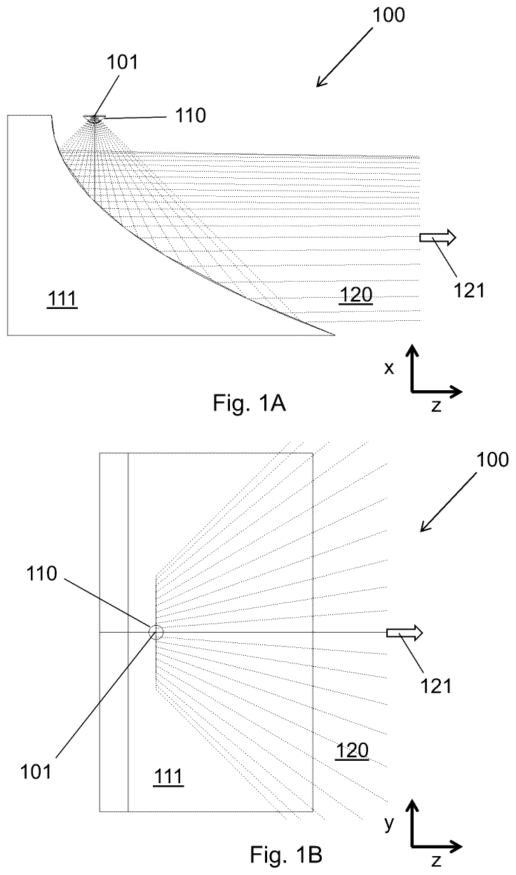

FIG. 1A and FIG. 1B show a schematic side view and a top view, respectively, of a first embodiment (1D-collimated beam configuration) of a device for air treatment 100, specifically an upper air germicidal UV (GUV) device, for generating a collimated UV radiation beam in one dimension (x-direction) and a defined wide angle distribution in the other (y-direction). The GUV device 100 comprises a UV radiation source 101, a primary lens 110 and an internal reflector 111. The lens 110 and the internal reflector 111 are elements of a collimating optics for collimating the UV radiation to form a beam 120 such that its rays are approximately parallel to each other and to the main direction of the propagating beam 120 when perpendicularly projected into the x-z-plane (see FIG. 1A). The lens 110 is configured to collect and pre-collimate the UV radiation of the LED source 101 to ensure that as much of the UV radiation as possible impinges on the internal reflector 111.

The GUV device 100 may further comprise elements that, for the sake of clarity, are not shown in FIGS. 1A, 1B such as a power supply for the UV radiation source 101, a frame for mounting the other elements, a housing, etc.

The UV radiation source 101 has a small etendue and may preferably be a UV-C LED. The LED source 101 including the primary lens 110, which is attached to the housing of the LED chip, is small compared to the internal reflector 111. Therefore, by approximation, the LED source 101 in combination with the beam-shaping primary lens 110 may be considered optically in the x-z-plane shown in FIG. 1A as a point radiation source. The internal reflector 111 is configured to have a parabolic shape in the x-z-plane and along the y-direction. Viewed in the x-z-plane (see FIG. 1A) the LED source 101 including the attached primary lens 110 is arranged near the focus point of the internal reflector 111. Therefore, the internal reflector 111 shapes the UV radiation coming from the LED source 101 and the primary lens 110 to form, by means of reflection, a beam 120 collimated in the x-direction (see FIG. 1A). Viewed in the y-z-plane (see FIG. 1B) the LED source 101 including the attached primary lens 110 is arranged in or near the center of the longitudinal extension of the reflector 111 in y-direction. The primary lens 110 is also configured to determine the wide opening angle of the UV radiation beam 120 in the second dimension along the y-direction, i.e. when viewed perpendicularly to the y-z-plane (see FIG. 1B).

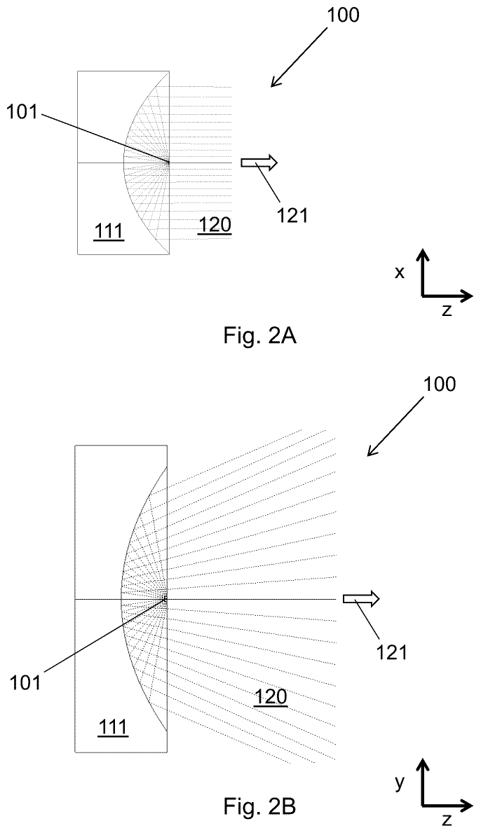

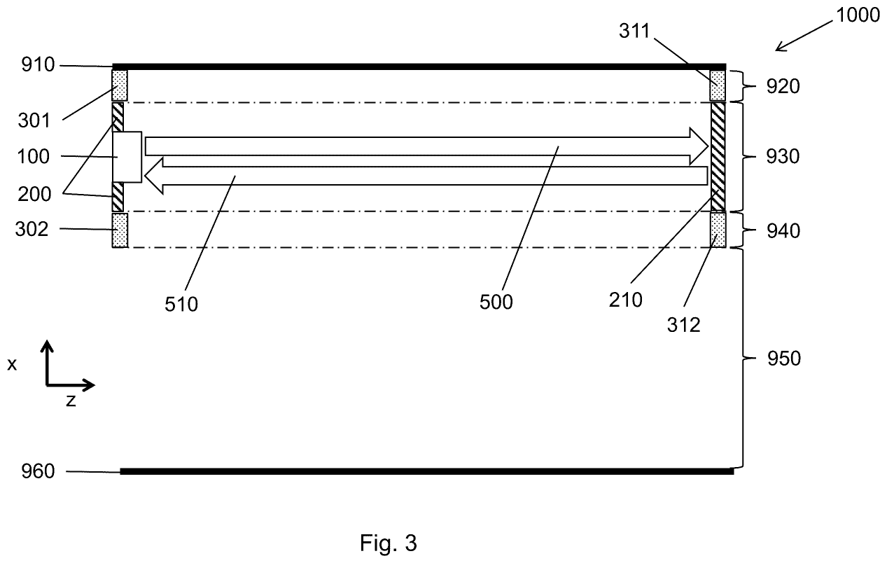

FIG. 2A and FIG. 2B show a schematic side view and a top view, respectively, of a second embodiment of a device for air treatment 100. In this embodiment, the LED source 101 is arranged in the focus of an anamorphic internal reflector 111. An additional primary lens is not needed. Instead, the UV radiation coming directly from the LED source 101 is shaped by the anamorphic internal reflector 111 to form, by means of reflection, a beam 120 collimated in the x-direction (see FIG. 2A), i.e. a result similar to FIG. 1A. For this purpose, the reflector has a suitable shape in the x-z-plane, e.g. mainly parabolic or elliptical. In the latter case, the shape of the internal reflector may also be adjusted such that the beam is not collimated across a room, but that the second focus is in a distance, e.g. 5 m to 10 m, where an additional similar external reflector is arranged to enable multiple passes of the UV radiation beam across the active zone in a room (see FIGS. 3-7). In the y-z-plane the anamorphic internal reflector 111 is formed to shape a wide opening angle of the rays of the UV radiation beam 120 when perpendicularly projected in the y-z-plane (see FIG. 2B), i.e. a result similar to FIG. 1B.

Other anamorphic setups are also feasible like anamorphic TIR-lenses, lens and cylindrical lens combinations, rectangular rods, extruded CPC (Compound Parabolic Concentrator) shaped reflectors, and others.

For a two-dimensional collimation, i.e. collimation of the UV radiation in the x-z-plane as well as in the y-z-plane, the usual setups of two to three lenses, TIR-lenses, parabolic or elliptical reflectors and so on are usable. More details can be found, for example, in “Nonimaging Optics” (Winston, R., Welford, W. T., Min□ano Juan C., &amp; Beni\acute{ } tez Pablo, Elsevier Academic Press, 2005) and in “Optical design of a freeform TIR lens for led streetlight” (Jinbo Jiang, Sandy To, W. B. Lee, Benny Cheung, Optik, 121(19), 1761-1765, 2010, https://doi.org/10.1016/1.iileo.2009.04.009).

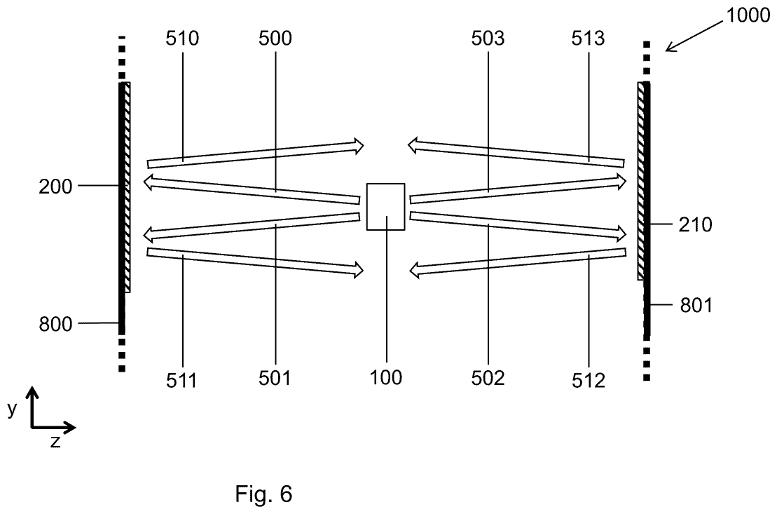

In another embodiment (not shown) the GUV device is configured to emanate more than one UV radiation beam (cf. FIG. 6). For example, with reference to FIG. 1B, two or more UV radiation sources may be arranged along the focal line of the reflector 111 in appropriate mutual distance. Alternatively, the GUV device may comprise two or more separate UV radiation beam units, each comprising a UV radiation source and dedicated beam-shaping and collimating optics.

FIG. 3 shows a schematic side view of a first embodiment of an assembly for air treatment 1000, located in an upper part of a room below the ceiling 910. At one end of the assembly 1000 an upper-air GUV device 100, particularly one according to FIG. 1A, 1B or 2A, 2B, is arranged (1D-collimated beam configuration). Therefore, in the x-direction of FIG. 3, i.e. when viewed in the horizontal viewing direction of the room, the UV radiation beam 500, emerging from the GUV device 100, is approximately collimated. More precisely, the beam angle of the UV radiation beam 500 is small, typically smaller than 2°, preferably smaller than 1° or even smaller than 0.5° to vertically keep the UV radiation within the active zone 930. In the y-direction, i.e. when viewed in the vertical viewing direction of the room, the beam-angle may vary, depending on the respective embodiment of the GUV device 100 (cf. FIGS. 1B, 2B).

At the other end of the assembly 1000 an external (first) reflector 210 is arranged. The external reflector 210 is configured to reflect the UV radiation beam 500 towards the GUV device 100. The backwards reflected beam 510 passes the active zone 930 a second time, but in reverse longitudinal direction. By this means, the distance between the GUV device 100 and the external reflector 210 defines the longitudinal extension of the active zone 930, i.e. in the z-direction shown in FIG. 3. The backwards reflected beam 510 may again be reflected by an additional (second) external reflector 200. Due to the multiple passes of back and forth reflections enabled by one or more external reflectors 200, 210, the UV irradiation in the active zone 930 is enhanced.

As already mentioned above, the optical configuration of the GUV device 100, i.e. the collimation of the UV radiation in the vertical plane of the room, and of the external reflectors 200, 210 is designed to confine the UV radiation within the active zone 930, thereby improving the efficiency of air treatment and establishing a reliable safe zone 950 in the lower part of the room as well. Safety of the safe zone 950 is even further improved by arranging beam-dumps 301, 302, 311, 312 at both ends of the assembly 1000, thereby defining transition zones 920, 940. By this means, residual UV stray radiation traveling outside the active zone 930 is absorbed by the beam-dumps 301, 302, 311, 312. The beam-dumps 301, 302 are arranged at the end of the assembly 1000 where the GUV device is arranged, and the beam-dumps 311, 312 are arranged where the external reflector 210 is arranged. Due to these measures the safe zone 950 extends from the floor of the room up to the lower transition zone 940.

Contrary to conventional setups, the ceiling 910 and walls (not shown) of a room, in which the assembly for air treatment 1000 is installed, are not hit by a major amount of the UV radiation, but only a small amount of stray radiation might reach the ceiling and walls. Since the UV stray radiation is absorbed by beam-dumps 301, 311 in the transition zone 920 below the ceiling 910, it is possible to mount further installations like luminaires, air cons, smoke detectors, etc. in a gap between the upper transition zone 920 and the ceiling 910 (not shown in FIG. 3).

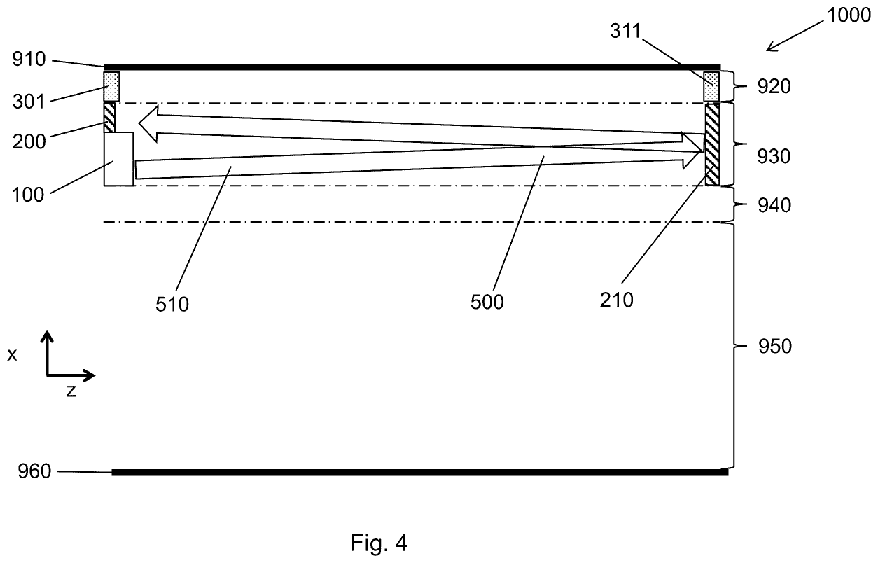

FIG. 4 shows a schematic side view of a second embodiment of an assembly for air treatment 1000. It is similar to the assembly shown in FIG. 3. The main difference lies in that the UV radiation beam 500 emanating from the GUV device 100 and the beam 510 reflected by the first external reflector 210 are both slightly tilted towards the ceiling 910. By this means, the safe zone 950 can be established without beam dumps in the transition zone 940 below the active zone 930. In this example, beam-dumps 301,311 are only arranged in the transition zone 920 above the active zone 930 and towards the ceiling 910. The tilt of the UV beams may be implemented by appropriate configuration or alignment of the upper-air GUV device 100 or the first external reflector 210, or a combination of these measures. The second external reflector 200 may also be adjusted appropriately to reflect the UV beam 510 for the third time back through the active zone 930 slightly tilted upwards to the ceiling 910.

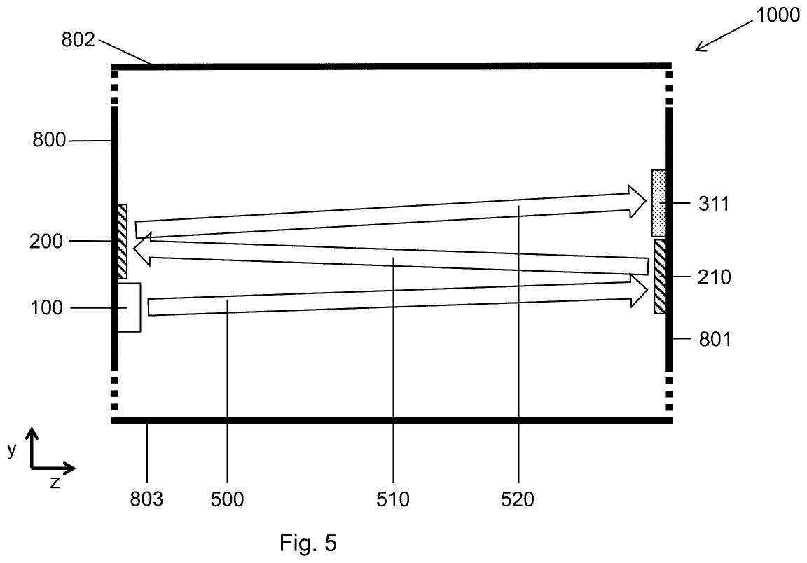

FIG. 5 shows a schematic top view of a third embodiment of an assembly 1000 for air treatment configured to implement a collimated beam in both the x-direction (horizontal viewing direction) and the y-direction (vertical viewing direction) of a room (2D-collimated beam configuration). The collimation can also be anamorphic, e.g., the collimation angle in the x-direction may be 1° and the collimation in the y-direction may be larger, e.g., 5°.

The GUV device 100 emits the initial UV radiation beam 500 which, in this embodiment, is reflected two times by the two external reflectors 210 and 200, resulting in the reflected beams 510 and 520. Additional external mirror areas for more than two reflections may be provided. At the end of this reflection-chain the UV radiation is absorbed by a beam-dumb 311. The GUV device 100, the external reflectors 200, 210 and beam-dump 311 are installed on opposite walls 800 and 801.

The simplest way to extent the active zone in the y-direction is to elongate the mirror area 210 and/or 200 accordingly, e.g. strip-shaped, and move the beam-dump 311 to the down-stream end of the multiple reflection passes.

In an alternative embodiment (not shown), the active zone is further extended in the y-direction by installing several of the assemblies 1000 shown in FIG. 5 next to each other or in an overlapping manner.

In another alternative embodiment (not shown), the same reflector and beam-dump components may be used by several upper-air GUV devices.

FIG. 6 shows a schematic partial top view of a fourth embodiment of an assembly 1000 for air treatment installed between opposite walls 800, 801, for example mounted hanging below the ceiling (not shown). Here the GUV device 100 is configured to emanate four 2D-collimated UV radiation beams 500 to 503, two beams 500, 501 towards the one wall 800 (to the left of the GUV device 100) and two beams 502, 503 towards the other wall 801 (to the right of the GUV device 100). The UV radiation beams 500-503 are reflected by two strip-shaped external reflectors 200, 210 each one installed on one of the opposite walls 800 and 801, respectively. The reflected UV radiation beams 510-513 are directed across the room towards the respective opposite wall. Beam-dumps installed at the respective downstream ends of each UV radiation beam reflection-chain are not shown in this partial top view.

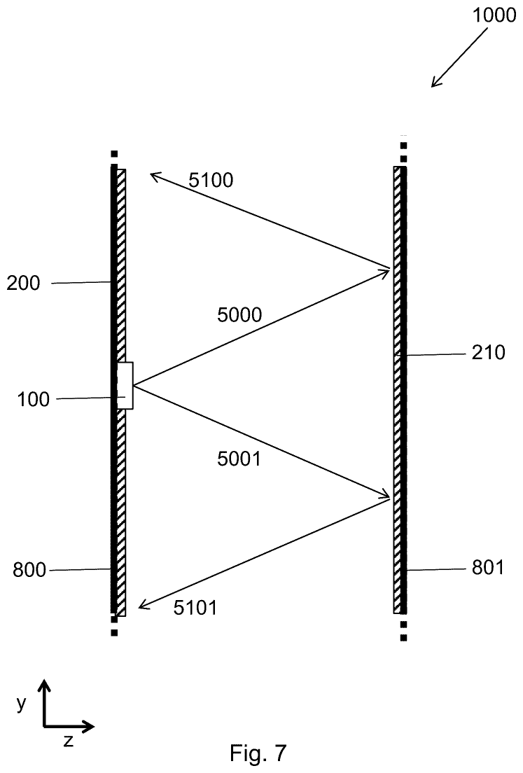

FIG. 7 shows a schematic partial top view of a fifth embodiment of an assembly 1000 for air treatment installed between opposite walls 800, 801. The GUV device 100 and the second external reflector 200 are mounted on the first wall 800. The first external reflector 210 is mounted on the second wall 800, opposite to the first wall 800. The GUV device 100 is configured to emanate a 1D-collimated beam. The collimation axis extends in the x-direction, i.e. it is perpendicular to the drawing plane and in the vertical direction of the room. In contrast to the assembly shown in FIG. 6, the GUV device 100 shown in FIG. 7 is configured to emit the UV radiation with respect to the y-direction in a wide angle, e.g., 10° to 80°, indicated by the two limiting rays 5000 and 5001 of the UV radiation beam bundle. Also shown are the limiting rays 5100 and 5101 reflected at the elongated first external reflector 210 back towards to the second external reflector 200. At the second external reflector 200 the UV rays are again reflected forth and towards the first external reflector 210 (not shown in this partial top view). This back and forth reflection may recur several times along the y-direction until the UV rays eventually end in a beam dump at the end of the reflection-chain (not shown).

Such wide-angle distributions of UV radiation may also be combined with an assembly with the upper-air GUV device positioned in the middle between the walls as shown in FIG. 6.

Beam dumps (not shown) may be mounted above and below the external reflector stripes or in dedicated places on the level of the external reflectors.

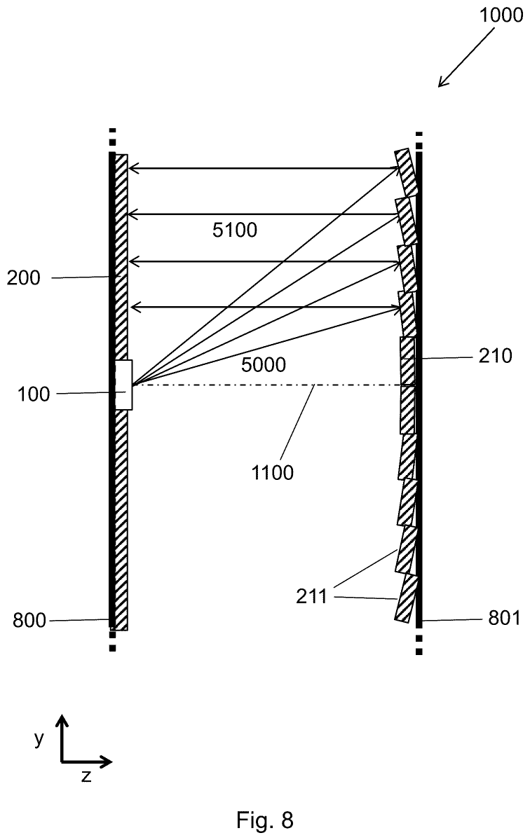

FIG. 8 shows a schematic partial top view of a sixth embodiment of an assembly 1000 for air treatment installed between opposite walls 800, 801. In this assembly the GUV device 100 is configured to emanate a 1D-collimated UV radiation beam similar to the configuration shown in FIG. 7, i.e. with a wide-angle distribution of UV radiation in the y-direction. Furthermore, the first external reflector 210, which is arranged vis-à-vis the GUV device 100, is of the Fresnel-type. The line 1100 indicates the symmetry plane of the external reflector 210. When viewed in the x-direction, i.e. perpendicular to the drawing plane, the UV radiation 5000 emitted by the GUV device 100 in a wide-angle distribution symmetrical to the line 1100 (indicated by a few exemplary rays 5000 only in the upper half relative to the symmetry line 1100) is reflected by the Fresnel structure 211 of the first external reflector 210 in a small-angle distribution (exemplary rays 5100) back to the opposite wall 800 and a flat second external reflector 200. The advantage of this assembly is that little radiation spills out beyond the area shown in FIG. 8, i.e. in the positive y-direction and the opposite negative y-direction.

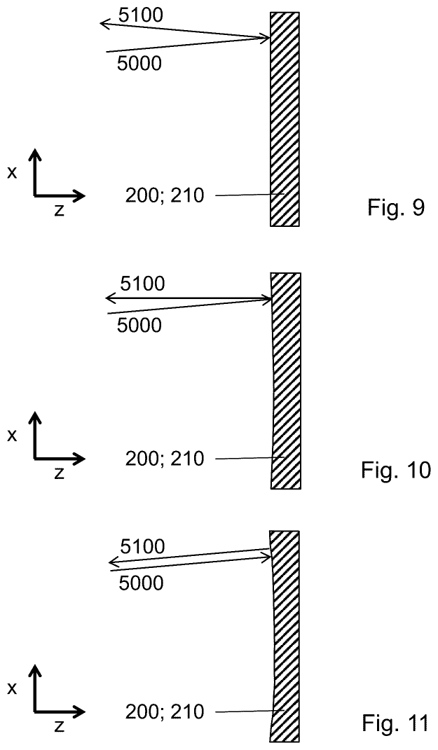

To make the most of the space of the active zone for distributing therein the UV radiation emanating from the GUV device different shapes of external reflectors in the x-z-plane of the assembly for air treatment are feasible. FIGS. 9 to 11 show three different examples for external reflectors 200, 210. Especially slightly curved mirror areas, like parabolic or elliptical shapes with a long focal length or focal point distance in the order of the distance between the external reflectors as shown in FIGS. 10, 11, respectively, are advantageous. However, also flat mirror areas as shown in FIG. 9 can be adequate external reflectors and have the advantage of easier feasibility.

In an alternative embodiment (not shown), the focal length of a curved reflector is adjustable by adjusting the curvature of the reflective surface of said reflector. For example, a flexible reflective sheet is fixed at the rim of the reflector frame. By mechanical, electrical, etc. means a variable pulling or pushing force in the center of the flexible reflective sheet may be established, thereby adjusting the curvature of the flexible reflective sheet and, consequently, the focal length of the curved reflector.

Some embodiments of the assembly for air treatment may comprise a combination of flat and curved external reflectors on either side.

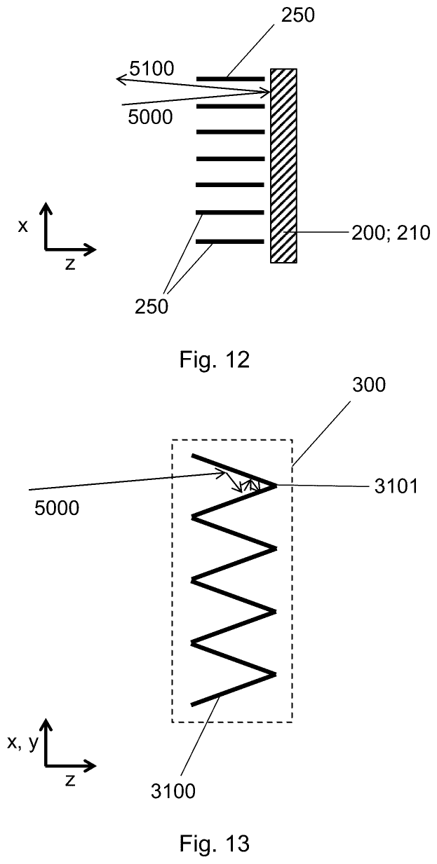

In a further refinement of the assembly for air treatment, a set of louvers 250 may be installed in front of the external reflectors 200, 210 as schematically shown in FIG. 12. The louvers 250 limit the reflecting angle thereby further improving the safety of the assembly. Due to this measure, less UV radiation can spill out of the active zone, even if the external reflectors 200, 210 produce more stray radiation caused by dust that may collect on the external reflectors over time.

FIG. 13 shows a schematic side or top view of a beam dump 300 (inside the dashed line) that essentially consist of a multi-W-shaped sheet structure 3100. The multi-W-shaped sheet structure 3100 is made of or coated with a material with a low reflectance for the harmful UV radiation, e.g., it may be made of an anodized metal sheet. Ideally, the structure 3100 should not scatter the radiation in large angles. One exemplary UV radiation ray 5000 is schematically shown that is reflected multiple times towards one of the tapered ends 3101 of the multi-W-shaped sheet structure 3100 and finally absorbed.

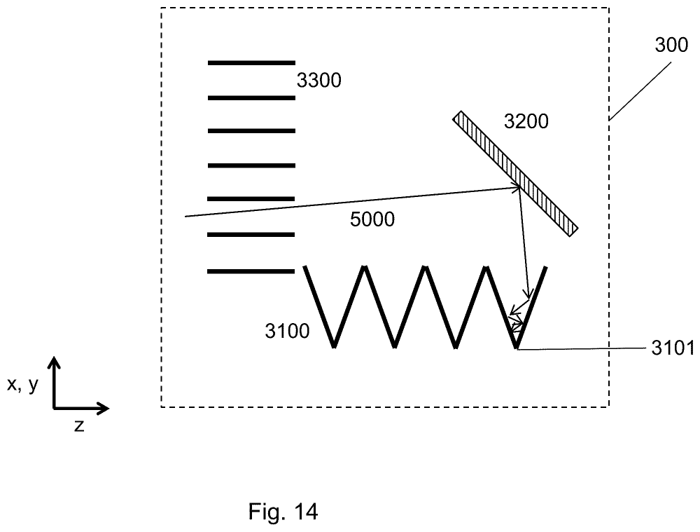

FIG. 14 shows a schematic side or top view of another example of a beam dump 300 (inside the dashed line). In addition to the W-shaped sheet structure 3100 shown in FIG. 13 it includes a louver system 3300 and a specular reflecting surface 3200. Like the W-shaped sheet structure 3100, the louver system 3300 is made of or coated with a material with a low reflectance for the harmful UV radiation.

The examples of a beam dump 300 shown in the FIGS. 13 and 14 may be used as beam dumps 301, 302, 311, 312 shown in FIGS. 3-5.

A device for air treatment is disclosed that is configured to emit a UV radiation beam at least approximately collimated in at least one dimension. The device for air treatment is arranged in an upper part of a room in such a manner that the rays of the UV radiation beam propagate approximately collimated when perpendicularly viewed in the horizontal viewing direction of the room. By means of this configuration and arrangement, an active zone, in which the UV radiation beam is vertically confined, is implemented in the upper part of the room.

1.-19. (canceled)

20. A device comprising:
at least one UV radiation source configured to emit radiation in a range of ultraviolet wavelengths; and
a beam-shaping and collimating optics configured to shape the radiation to a UV beam at least approximately collimated in at least one dimension,
wherein a beam angle, defined as an angle to an optical axis of the UV beam including 99% of a radiant flux is smaller than 2°.

21. The device of claim 20, wherein the UV radiation source and the beam-shaping and collimating optics are further configured to at least approximately collimate the radiation in a first direction of space perpendicular to a main direction of the UV beam.

22. The device of claim 21, wherein the UV radiation source and the beam-shaping and collimating optics are further configured to at least approximately collimate the radiation in a second direction of space perpendicular to the first direction of space and to the main direction of the UV beam.

23. The device of claim 21, wherein the UV radiation source and the beam-shaping and collimating optics are further configured to shape the UV beam to have a wide-angle distribution with respect to a second direction of space, perpendicular to the first direction of space and to the main direction of the UV beam.

24. The device of claim 20, wherein the UV radiation source is a source that has a small etendue.

25. The device of claim 20, wherein geometrical dimensions of an emitting area of the UV radiation source is smaller than 2 mm in at least one direction.

26. The device of claim 20, wherein the beam shaping and collimating optics comprises at least one lens or TIR-lens or at least one reflector or any combination thereof.

27. An assembly comprising:
at least one device of claim 20,
wherein the at least one device is configured for establishing an active zone for air treatment with the radiation in an upper part of a room,
wherein the at least one device is configured to be able to emit the UV beam such that inside the active zone the UV beam is at least approximately vertically collimated thereby confining a transversal extension of the active zone in a vertical direction of the room; and
at least one beam-dump configured to be able to collect and absorb the UV beam,
wherein the at least one beam-dump is arranged vis-à-vis the at least one device in a distance along a main direction of the UV beam thereby defining a longitudinal extension of the active zone in between.

28. An assembly comprising:
at least one device of claim 20,
wherein the at least one device is configured for establishing an active zone for air treatment with the radiation in an upper part of a room,
wherein the at least one device is configured to be able to emit the UV beam such that inside the active zone the UV beam is at least approximately vertically collimated thereby confining a transversal extension of the active zone aligned with a vertical direction of the room; and
at least one first external reflector arranged vis-à-vis the at least one device in a distance along a main direction of the UV beam thereby defining a longitudinal extension of the active zone in between,
wherein the at least one first external reflector is configured to be able to reflect the UV beam back into the active zone.

29. The assembly of claim 28, further comprising at least one second external reflector arranged vis-à-vis the at least one first external reflector at a boundary of the longitudinal extension of the active zone opposite to the at least one first external reflector.

30. The assembly of claim 29, wherein the at least one first external reflector and the at least one second external reflector are configured to be able to reflect the UV beam from one external reflector to another external reflector thereby passing through the active zone multiple times.

31. The assembly of claim 29, wherein the at least one first external reflector and the at least one second external reflector are configured to be able to reflect the UV beam to pass through the active zone in a zigzag-like manner.

32. The assembly of claim 29, further comprising at least one set of louvers arranged in front of the at least one first external reflector and/or the at least one second external reflector, wherein the at least one set of louvers is configured to limit the reflecting angle of the respective external reflector.

33. The assembly of claim 28, wherein the at least one first external reflector has a Fresnel structure.

34. The assembly of claim 28, further comprising at least one beam-dump configured to be able to collect and absorb the UV beam.

35. The assembly of claim 34, wherein the at least one beam-dump is arranged at a downstream end of the UV beam and/or arranged and configured to be able to absorb a residual UV radiation leaking from the active zone.

36. The assembly of claim 28, further comprising a fluorescent material arranged and configured to visually indicate UV radiation leaking from the active zone and/or indicating operation of the at least one device.

37. A method for air treatment in a room, the method comprising:
providing at least one device of claim 20; and
arranging the at least one device in the room such that an active zone, in which the UV beam emanating from the at least one device for is confined, is implemented in an upper part of the room,
wherein a collimation axis of the UV beam is at least approximately aligned with a vertical direction of the room.
